(19) United States
(12) Patent Application Publication
(10) Pub. No.: US 2007/0162098 A1
(43) Pub. Date: Jul. 12, 2007

(54) Prosthetic hearing implant electrode assembly having optimal length for atraumatic implantation

(75) Inventors: Frank Risi, Newtown (AU); Robert Briggs, Melbourne (AU); Martin Svehla, Botany (AU); Thomas Lenarz, Hannover (DE)

Correspondence Address: JAGTIANI + GUTTAG, 10363-A DEMOCRACY LANE, FAIRFAX, VA, 22030, US

(73) Assignee: Cochlear Limited, Lane Cove (AU)

(21) Appl. No.: 11/605,952

(22) Filed: Nov. 30, 2006

Related U.S. Application Data
(60) Provisional application No. 60/748,274, filed on Dec. 8, 2005.
(60) Provisional application No. 60/748,217, filed on Dec. 8, 2005.
(60) Provisional application No. 60/748,273, filed on Dec. 8, 2005.
(60) Provisional application No. 60/748,314, filed on Dec. 8, 2005.

(51) Int. Cl. A61N 1/05

(52) U.S. Cl. 607/137

(57) ABSTRACT

An elongate carrier member configured for implantation into a cochlea, the carrier member having a proximal end adapted to be positioned in a basal region of the cochlea, and a distal end adapted to be positioned in an apical region of the cochlea; and a plurality of electrodes disposed at the distal end of the carrier member; wherein the carrier member has a length such that the carrier member cannot be implanted in the cochlea beyond a maximum insertion depth at which residual hearing of the apical region of the cochlea is substantially preserved and at which said carrier member provides electrical-only stimulation to a depth in the cochlea of approximately 270° to 320°. In one embodiment, the carrier member has a length such that the carrier member has a maximum insertion depth into the cochlea of between approximately 14 mm and 18 mm.

### CROSS-REFERENCE TO RELATED APPLICATIONS

The present application claims priority from U.S. Provisional Patent Application 60/748,217 entitled “Promoting Curvature and Maintaining Orientation In An Electrode Carrier Member Of A Prosthetic Hearing Implant,” filed Dec. 8, 2005; U.S. Provisional Patent Application 60/748,273 entitled “Electrode Carrier Member Having An Embedded Stiffener For A Prosthetic Hearing Implant,” filed Dec. 8, 2005; and U.S. Provisional Patent Application 60/748,314 entitled “Electrode Carrier Member For A Prosthetic Hearing Implant Having Variable Pitch Electrodes To Facilitate Atraumatic Implantation,” filed Dec. 8, 2005, and U.S. Provisional Patent Application 60/748,274 entitled “Electrode Carrier Member for a Prosthetic Hearing Implant Having Optimal Length for Altraumatic Implantation,” filed Dec. 8, 2005, all of which are hereby incorporated by reference herein.

### BACKGROUND

1. Field of the Invention

The present invention relates generally to stimulating medical devices and, more particularly, to an implantable electrode assembly for a stimulating medical device.

2. Related Art

Hearing loss is generally of two types, namely conductive and sensorineural. The treatment of both of types of hearing loss has been quite different, relying on different principles to deliver sound signals to be perceived by the brain as sound. Conductive hearing loss occurs when the normal mechanical pathways for sound to reach the hair cells in the cochlea are impeded, for example, by damage to the ossicles. In such cases, hearing loss is often improved with the use of conventional hearing aids, which amplify the sound so that acoustic information reaches the cochlear hair cells. Such hearing aids utilize acoustic mechanical stimulation, whereby the sound is amplified according to a number of varying techniques, and delivered to the inner ear as mechanical energy. This may be through a column of air to the eardrum, or through direct delivery to the ossicles of the middle ear.

On the other hand, sensorineural hearing loss is due to the absence or destruction of the cochlear hair cells which are needed to transduce acoustic signals into auditory nerve impulses. Individuals suffering from this type of hearing loss are unable to derive any benefit from conventional hearing aid systems regardless of the volume of the acoustic stimulus. This is because the natural mechanisms for transducing sound energy into auditory nerve impulses are either absent or damaged. In such cases, cochlear™ implants (also referred to as cochlear™ devices, cochlear™ prostheses, cochlear™ implant systems, and the like; simply “cochlear implants” herein) have been developed to provide the sensation of hearing to such individuals. In cochlear implants, electrical stimulation is provided via stimulating electrodes positioned as close as possible to the nerve endings of the auditory nerve, essentially bypassing the hair cells in a normally functioning cochlea. The application of a stimulation pattern to the nerve endings causes impulses to be sent to the brain via the auditory nerve, resulting in the brain perceiving the impulses as sound.

More recently, there has been an increased interest in Electro-Acoustical Stimulation (EAS) in which electrical stimulation of the cochlea is used in conjunction with acoustical stimulation. It is relatively common in hearing impaired individuals to experience sensorineural hearing loss for sounds in the high frequency range, and yet still be able to discern sounds in the middle to low frequency range, through the use of a conventional hearing aid, or naturally. Traditionally, in the majority of such cases, the recipient would only receive treatment to preserve and improve the hearing for the middle to low frequency sounds, most probably via a conventional hearing aid, and little would be done to attempt to restore the hearing loss for the high frequency sounds. This is due to the potential trauma caused by the implantation of an electrode assembly into the cochlea. Only if the individual lost the ability to perceive middle to low frequency sounds would consideration then be given to restoring the hearing loss for the high frequency sounds, in which case a cochlear implant would be considered a possible solution.

### SUMMARY

In one aspect of the invention, an electrode assembly for use in a prosthetic hearing implant is disclosed, the electrode assembly comprising: an elongate carrier member for implantation into the cochlea, the carrier member having a proximal end adapted to be positioned in a basal region of the cochlea, and a distal end adapted to be positioned in an apical region of the cochlea; a plurality of electrodes disposed on the carrier member; wherein the carrier member has a length such that the carrier member has a maximum insertion depth into the cochlea of approximately 14 mm to approximately 18 mm.

In another aspect of the invention, an electrode assembly for use in a prosthetic hearing implant is disclosed, the electrode assembly comprising: an elongate carrier member configured for implantation into a cochlea, the carrier member having a proximal end adapted to be positioned in a basal region of the cochlea, and a distal end adapted to be positioned in an apical region of the cochlea; and a plurality of electrodes disposed on said carrier member; wherein the carrier member has a length such that the carrier member cannot be implanted in the cochlea beyond a maximum insertion depth at which residual hearing of the apical region of the cochlea is substantially preserved and at which said carrier member provides electrical-only stimulation to a depth in the cochlea of approximately 270° to 320°.

### BRIEF DESCRIPTION OF THE DRAWINGS

Embodiments of the present invention are described herein with reference to the following figures, in which:

FIG. 1 is a perspective view of an implanted cochlear implant suitable for implementing embodiments of the present invention;

FIG. 2A is a side view of an electrode assembly in accordance with one embodiment of the present invention shown prior to insertion into a cochlea;

FIG. 2B is a top view of the electrode assembly illustrated in FIG. 2A, shown after insertion into a cochlea;

FIG. 2C is a cross sectional view of the electrode assembly of FIGS. 2A and 2B taken along section line 2C-2C in FIG. 2A;

FIG. 2D is a cross sectional view of the electrode assembly of FIGS. 2A and 2B taken along section line 2D-2D in FIG. 2A; and

FIG. 2E is a cross sectional view of the electrode assembly of FIGS. 2A and 2B taken along section line 2E-2E in FIG. 2A.

### DETAILED DESCRIPTION

Embodiments of the present invention are generally directed to an apparatus and method for facilitating implantation of a medical device into a patient (referred to herein as a recipient). Embodiments of the present invention are described below in connection with one type of medical device, a prosthetic hearing implant and, more specifically, a cochlear implant. Cochlear implants use direct electrical stimulation of auditory nerve cells to bypass absent or defective hair cells that normally transduce acoustic vibrations into neural activity. Such devices generally use multi-contact electrodes inserted into the scala tympani of the cochlea so that the electrodes may differentially activate auditory neurons that normally encode differential pitches of sound. Such devices are also used to treat a smaller number of patients with bilateral degeneration of the auditory nerve. For such patients, the cochlear implant provides stimulation of the cochlear nucleus in the brainstem. Such devices, therefore, are commonly referred to as auditory brainstem implants (ABIs).

Exemplary embodiments of a cochlear implant include a Contour™, Freedom™, Nucleus™ or Cochlear™ implant sold by Cochlear Limited, Australia. Such devices are described in U.S. Pat. Nos. 4,532,930, 6,537,200, 6,565,503, 6,575,894, and 6,697,674, the entire contents and disclosures of which are hereby incorporated by reference herein. It should be understood to those of ordinary skill in the art that embodiments of the present invention may be used in other stimulating medical devices such as neurostimulators, cardiac pacemakers/defibrillators, etc. as well as other medical devices which utilize an elongate carrier member to temporarily or permanently implant, deliver or otherwise introduce a therapeutic agent, sensor, device, etc. into a recipient.

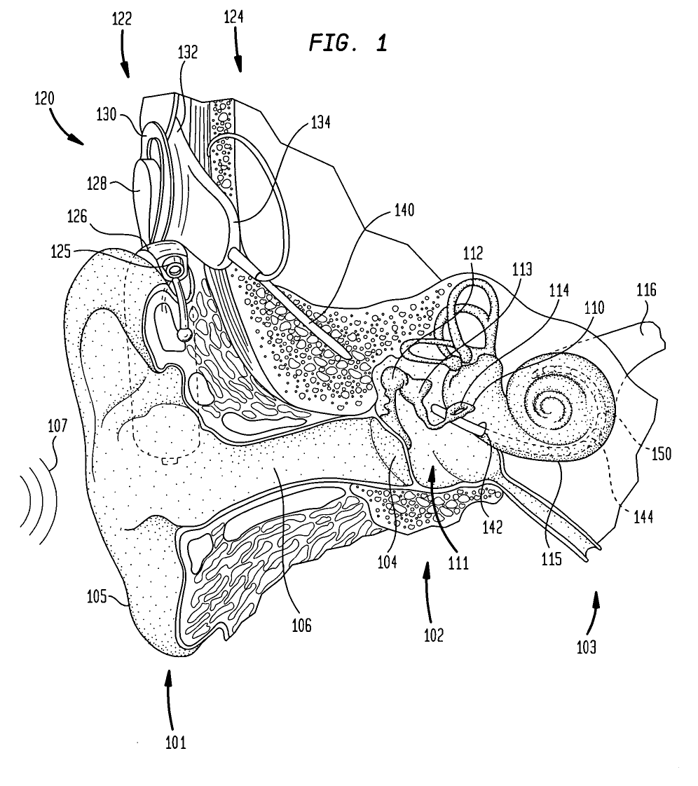

FIG. 1 is a cut-away view of the relevant components of outer ear 101, middle ear 102 and inner ear 103, which are described next below. In a fully functional ear, outer ear 101 comprises an auricle 105 and an ear canal 106. An acoustic pressure or sound wave 107 is collected by auricle 105 and channeled into and through ear canal 106. Disposed across the distal end of ear cannel 106 is a tympanic membrane 104 which vibrates in response to acoustic wave 107. This vibration is coupled to oval window, or fenestra ovalis, 110 through three bones of middle ear 102, collectively referred to as the ossicles 111.

Ossicles 111 comprises the malleus 112, the incus 113 and the stapes 114. Bones 112, 113 and 114 of middle ear 102 serve to filter and amplify acoustic wave 107, causing oval window 110 to articulate, or vibrate. Such vibration sets up waves of fluid motion within cochlea 115. Such fluid motion, in turn, activates tiny hair cells (not shown) that line the inside of cochlea 115. Activation of the hair cells causes appropriate nerve impulses to be transferred through the spiral ganglion cells (not shown) to auditory nerve 116 and, ultimately, to the brain where they are perceived as sound. In some persons experiencing sensorineural hearing loss, there is an absence or destruction of the hair cells. Cochlear implant 120 is utilized to directly stimulate the ganglion cells to provide a hearing sensation to such persons.

FIG. 1 also shows how cochlear implant 120 is positioned in relation to outer ear 101, middle ear 102 and inner ear 103. Cochlear implant 120 comprises external component assembly 122 which is directly or indirectly attached to the body of the recipient, and an internal component assembly 124 which is temporarily or permanently implanted in the recipient. External assembly 122 comprises microphone 125 for detecting sound which is provided to a behind-the-ear (BTE) speech processing unit 126 that generates coded signals. The coded signals are provided to an external transmitter unit 128, along with power from a power source (not shown) such as a battery. External transmitter unit 128 comprises an external coil 130 and, preferably, a magnet (not shown) secured directly or indirectly in external coil 130.

Internal component assembly 124 comprises an internal receiver unit 132 having an internal coil (not shown) that transcutaneously receives power and coded signals from external assembly 122, and provides such signals to a stimulator unit 134. In response to the coded signals, stimulator 134 applies stimulation signals to cochlea 115 via an implanted electrode assembly 140. Electrode assembly 140 enters cochlea 115 via a cochleostomy 142 or through oval window 110, and has an array 144 of one or more electrodes 150 positioned to be substantially aligned with portions of tonotopically-mapped cochlea 115. The delivery of stimulation signals at various locations along cochlea 115 causes a hearing percept representative of the received sound 107.

While cochlear implant 120 is described as having external components, in another embodiment, the controller, including the microphone, speech processor and power supply, may also be implantable. In such embodiments, the controller may be contained within a hermetically sealed housing or the housing used for stimulator unit 134.

Electrode assembly 140 preferably assumes an optimal electrode position in cochlea 115 upon or immediately following implantation into the cochlea. It is also desirable that electrode assembly 140 be configured such that the insertion process causes minimal trauma to the sensitive structures of cochlea 115. Usually electrode assembly 140 is pre-curved, held in a straight configuration at least during the initial stages of the implantation procedure, conforming to the natural shape of the cochlea during and subsequent to implantation.

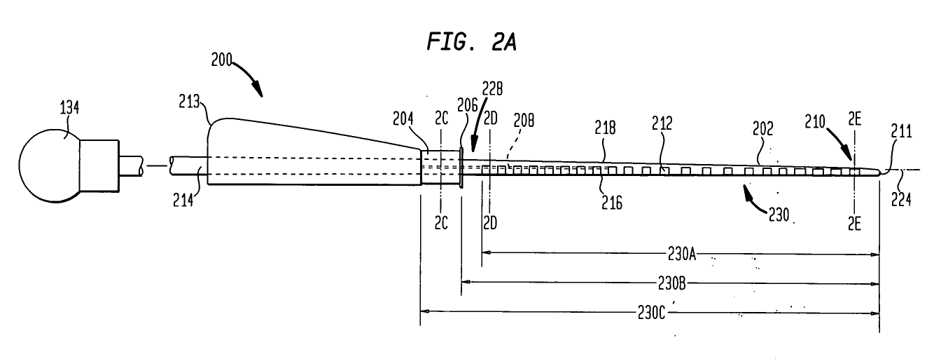

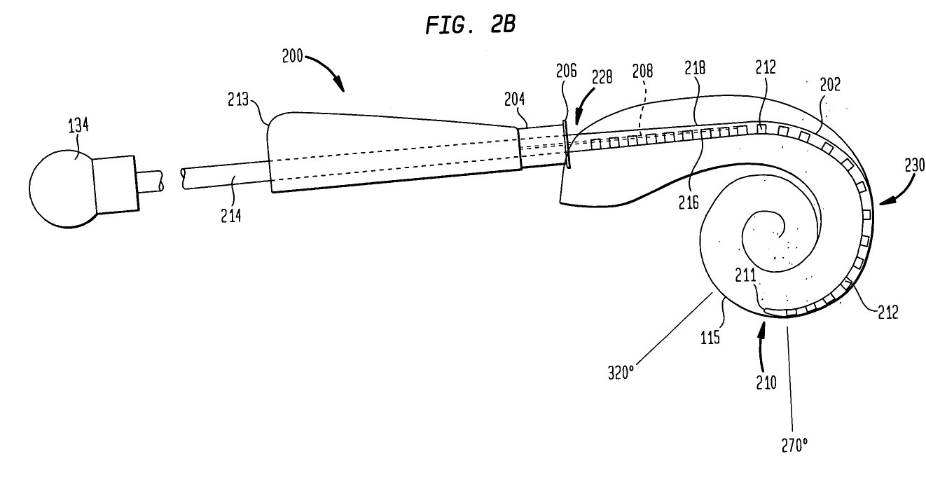

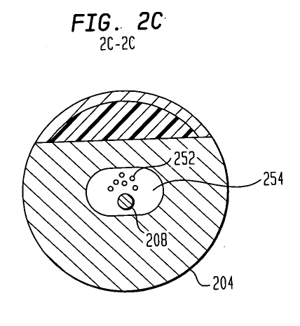

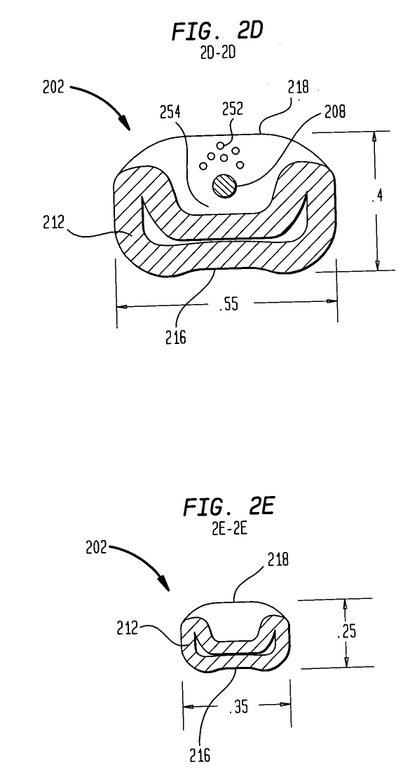

FIGS. 2A and 2B are side views of one embodiment of electrode assembly 140, referred to herein as electrode assembly 200. In FIG. 2A, electrode assembly 200 is shown in its configuration prior to insertion into cochlea 115, while in FIG. 2B electrode assembly 200 is shown after insertion into cochlea 115. FIGS. 2C-2E are cross-sectional views of electrode assembly 200 taken along their respective section lines in FIG. 2A.

Electrode assembly 200 comprises a carrier member 202, a collar member 204, a holding member 213 and one or more leads 214. Carrier member 202 has a distal end 210 and a proximal end 228 connected to the distal end of laterally-extending collar member 204. The opposing proximal end of collar member 204 is connected to holding member 213. Lead 214 physically and electrically connects electrode assembly 200 and electrodes 212 disposed thereon with receiver/stimulator unit 134.

When implanted in a recipient, the surface of carrier member 202 which faces the interior of cochlea 115 is referred to herein as the medial surface 216 of carrier member 202. The opposing side of carrier member 202, referred to herein as lateral surface 218, faces the external wall and bony capsule (not shown) of cochlea 115. It should be understood that the terms medial surface, medial direction, and the like, are generally used herein to refer to the surfaces, features and directions toward the center of cochlea 115, while the terms lateral surface, lateral direction, and the like, are generally used herein to refer to surfaces, features and directions toward the exterior of cochlea 115.

A plurality of spaced-apart electrodes 212 are mounted on or in carrier member 202. The array of electrodes 212 is referred to herein as electrode array 230. Electrodes 212 may be disposed in a linear or non-linear array 230 on or in carrier member 202, and may be positioned to align with predetermined regions of tonotopically mapped cochlea 115. In alternative embodiments, electrodes 212 are implemented as described in the U.S. Provisional Patent Applications 60/748,217, 60/748,273 and 60/748,314, which are hereby incorporated by reference herein. Such arrangements allow for individual electrodes 212 to be utilized to stimulate selected regions of cochlea 115.

In one embodiment, electrodes 212 are half-band electrodes disposed in or on medial surface 216 of carrier member 202. It should be appreciated, however, that any electrodes now or later developed suitable for a particular application may be used in alternative embodiments of the invention. For example, in one alternative embodiment, electrodes 212 are banded electrodes extending substantially around the circumference of carrier member 202. In another embodiment, electrodes 212 do not laterally extend to or around the edges of carrier member 202. Typically, each electrode 212 is arranged such that its exposed surface is substantially parallel to a longitudinal axis 224 of carrier member 202. It should be appreciated, however, that other locations and orientations may be implemented in alterative embodiments. It should further be appreciated that the quantity of electrodes 212 may vary from as few as one or two to as many as twenty-four or more.

In certain embodiments, at least one electrode 212 has a surface that is at least adjacent medial surface 216 of carrier member 202. Preferably, one or more electrodes 212 has a surface that is collocated with medial surface 216 of carrier member 202. In another embodiment, the surfaces of electrodes 212 are raised above or recessed into medial surface 216 of carrier member 202.

Electrodes 212 may be manufactured from a biocompatible conductive material such as platinum, although other materials or combinations of materials may be used. Alternatively, electrodes 212 may be coated with a biocompatible covering that does not interfere with transfer of stimulation signals to cochlea 115.

Each electrode 212 is electrically connected to at least one multi- or single-filament wire 252 (FIGS. 2C and 2D) that is embedded within flexible carrier member 202, collar member 204, handle member 213 and lead 214. In one embodiment, wires 252 are embedded in a volumetric core 254 of carrier member 202 and collar member 204. In an alternative embodiment, wires 252 may be located at or near surface 216 and/or surface 218 of carrier member 202. In other embodiments, wires 252 are embedded in different regions of carrier member 202 to facilitate curvature or to maintain orientation of carrier member 202 once it is implanted. It is through wires 252 that stimulator/receiver unit 134 (FIG. 1) provides electrical stimuli to selected electrodes 212. In one embodiment, wires 252 are connected to electrodes 212 by welding, although any suitable electrical connections now or later developed may be used.

It should be appreciated that the quantity of wires 252 connected to each electrode 212 may vary. For example, in one embodiment, at least two electrically conducting wires 252 are connected to each electrode 212. It should also be appreciated that suitable transmission means other than wires may be used to communicably couple receiver/stimulator unit 134 with electrodes 212. For example semiconductors or wireless technologies may be used.

In one embodiment, lead 214 extends from handle member 213 to stimulator 134 or at least the housing thereof. In one particular embodiment, lead 214 is continuous with no intermediate electrical connectors external the housing of stimulator unit 134; that is, there are no external connectors required to electrically connect electrode assembly 200 to stimulator 134. One advantage of this arrangement is that there is no requirement for a surgeon implanting electrode assembly 200 to make the necessary electrical connection between wires 252 extending from electrodes 214 and stimulator 134. Stimulator 134 is preferably encased within a housing that is implantable within the recipient, such as within a recess in the bone behind the ear posterior to the mastoid.

Returning to FIG. 2A, holding member 213 is configured to provide improved manual control of electrode assembly 200, and to identify electrode orientation. In alternative embodiments, holding member 213 may be configured as described in U.S. patent application Ser. No. 10/825,360, which is hereby incorporated by reference herein.

In certain embodiments, a profiled tip or tip region 211 is used to guide electrode assembly 200 during implantation in a manner that reduced friction. Alternative embodiments of tip region 210 are described in International Application PCT/US06/34010 entitled, “Elongate Implantable Carrier Member Having An Embedded Stiffener,” and filed Aug. 31, 2006; U.S. patent application entitled “Flexible Electrode Assembly Having Variable Pitch Electrodes for a Stimulating Medical Device,” filed concurrently under Attorney Docket Number: COCH-0181-UT1; and U.S. patent application entitled “Promoting Curvature and Maintaining Orientation of an Electrode Carrier Member of a Stimulating Medical Device,” filed concurrently under Attorney Docket Number: COCH-0178-UT1 all of which are hereby incorporated by reference herein. In alternative embodiments, tip region 210 may be as described in U.S. patent application Ser. Nos. 10/825,358 and 11/125,171, which are also hereby incorporated by reference herein.

In certain embodiments, carrier member 202 also includes a stiffening member 208 as described in International Application. No. PCT/US06/34010; filed Aug. 31, 2006, which is hereby incorporated by reference herein.

Collar member 204 may serve as a region for grasping electrode assembly 200. Preferably, collar member 204 is constructed and arranged to prevent insertion of carrier member 202 beyond a predetermined maximum depth. This reduces the risk of the surgeon over-inserting electrode assembly 200, which could cause trauma to the delicate structures of cochlea 115. In one preferred embodiment, the predetermined maximum insertion depth 230B is approximately 16 mm+/−2 mm, which is described in further detail below.

In the embodiment shown in FIGS. 2A-2E, collar member 204 has a diameter greater than that of carrier member 202. As one of ordinary skill in the art should appreciate, the configuration, orientation and dimensions of collar member 204 can vary depending on the intended implant location and, more generally, on the application of electrode assembly 200. In one embodiment, collar member 204 extends substantially at right angles to longitudinal axis 224 of carrier member 202. In one embodiment, collar member 204 has a length of between about 1.5 mm and about 2.0 mm. In another embodiment, collar member 204 has a length of between about 1.5 mm and about 2.5 mm. Alternatively, collar member 204 may be constructed and arranged generally as described in U.S. patent application Ser. Nos. 10/518,811 and 11/125,171, which are hereby incorporated by reference herein.

In the embodiments illustrated herein, a band 206 is located adjacent collar member 204, circumferentially surrounding carrier member 202 at the junction of carrier member 202 and collar member 204. Band 206 is formed of a material designed to produce a tissue reaction that facilitates tissue attachment to the band, promoting sealing of cochlea 115. In one embodiment, this material primarily contains platinum. This is especially important in preserving residual hearing as prompt sealing of cochlea 115 may greatly reduce the chances of infection of inner ear 103 due to implantation of electrode assembly 200.

Referring to FIG. 2A, in some embodiments, the length 230B of carrier member 202 is between approximately 14 mm and 18 mm, and preferably, approximately 16 mm. The inventors have found that this dimension is optimal for this application where the preservation of residual hearing is desired. In such an application, electrode carrier member 202 is of a specific length such that it can be inserted with minimal friction to a length that ensures the basal region (high frequencies) can be stimulated. However if residual hearing is lost due to the procedure or naturally over time, the electrode length is sufficient to provide the same benefits as current cochlear implants without having to undergo replacement surgery.

Indications for implantation of cochlear implants have historically been profound to severe hearing loss. However recent studies have been undertaken to assess the benefits of implantation of candidates with some level of residual hearing, typically in the lower frequency spectrum, but who have profound hearing loss in the higher frequency spectrum. These candidates typically fall outside the current indications as they still have a significant level of residual hearing, however their performance with hearing aids alone is generally poor. Current electrode assembly designs do not provide for atraumatic implantation into the cochlea, and therefore do not generally preserve the residual hearing that is still present.

Unfortunately, current straight electrode designs generally utilize the fragile structures of the cochlea to guide and curve the electrode. Due to frictional forces and the radius of curvature, however, these forces increase the further the electrode is inserted, therefore significantly risking intra-cochlear trauma, and therefore risking loss of any residual hearing.

Embodiments of the electrode assembly of the present invention attempt to overcome these drawbacks by striking a balance between depth of insertion to provide a very high confidence of preserving residual hearing, while also being long enough to provide adequate electrical-only stimulation if residual hearing is lost for whatever reason. This length has been determined to be approximately 16 mm+/−2 mm. Experiments on force transducer machines with plastic cochlear models, and insertions into temporal bones have shown that an electrode of this length can be inserted fully in one single insertion with minimal friction (that is, to the point just before the insertion forces become significant, risking intra-cochlear trauma), however still reaching a depth of approximately 270° to 320°, as shown in FIG. 2B, so as still to provide significant overlap and therefore adequate electrical-only stimulation.

In certain embodiments, electrode carrier member 202 is longitudinally-tapered as shown in FIGS. 2A and 2B. As such, the dimensions of carrier member 202 at section line 2D-2D are greater that the dimensions of carrier member 202 at section line 2E-2E, as shown in FIGS. 2D and 2E. Such a tapered configuration may be continuous, gradual tapering carrier member 202 to tip region 211. Preferably, tip region 211 is configured to facilitate the insertion of carrier member 202 into a recipient's cochlea 115. In one embodiment, tip member region 211 comprises a taper which slopes from lateral surface 218 rearward and inward toward medial surface 216. Such a tapered tip region 211 aids the coiling of carrier member 202 and further helps prevent damage to the delicate structures of cochlea 115.

Although the present invention has been fully described in conjunction with several embodiments thereof with reference to the accompanying drawings, it is to be understood that various changes and modifications may be apparent to those skilled in the art. Such changes and modifications are to be understood as included within the scope of the present invention as defined by the appended claims, unless they depart therefrom.

It should be understood that embodiments and features of the present invention may be combined with any other features or embodiments described in the documents attached hereto and incorporated by reference herein as long as such combinations are possible and non-conflicting.

All documents, patents, journal articles and other materials cited in the present application are hereby incorporated by reference.

What is claimed is:

1. An electrode assembly for use in a prosthetic hearing implant, comprising:
an elongate carrier member for implantation into the cochlea, the carrier member having a proximal end adapted to be positioned in a basal region of the cochlea, and a distal end adapted to be positioned in an apical region of the cochlea;
and a plurality of electrodes disposed on said carrier member;
a laterally-extending collar member connected to said proximal end of said carrier member;
wherein said carrier member has a length such that said carrier member has a maximum insertion depth into the cochlea of approximately 14 mm to approximately 18 mm.

2. The electrode assembly of claim 1, wherein said carrier member has a length such that said maximum insertion depth of approximately 16 mm.

3. The electrode assembly of claim 1, further comprising:
a laterally-extending collar member connected to said proximal end of said carrier member and configured to abut the cochlea when said carrier member is implanted at said maximum insertion depth, and to prevent further insertion of said carrier member into the cochlea.

4. The electrode assembly of claim 1, wherein said carrier member is longitudinally-tapered, wherein said proximal end of said carrier member has greater dimensions than said distal end of said carrier member.

5. The electrode assembly of claim 4, wherein in one or more radial directions said collar member has a diameter greater than a diameter of said carrier member.

6. The electrode assembly of claim 4, wherein said collar member has a length of between about 1.5 mm and about 2.5 mm.

7. The electrode assembly of claim 3, further comprising:
a band circumferentially surrounding said proximal end of said carrier member, said band formed of a material that produces a tissue reaction that facilitates tissue attachment to the band.

8. The electrode assembly of claim 1, wherein said carrier member has opposing medial and lateral longitudinal surfaces, wherein when implanted, said medial surface faces toward the center of the cochlea and said lateral surface that faces toward the exterior of the cochlea, wherein said electrode assembly further comprises:
a tapered tip region that slopes from said distal end toward said proximal end, and from said lateral surface inward toward said medial surface.

9. The electrode assembly of claim 3, further comprising:
a holding member having a distal end connected to said proximal end of said collar member, wherein said holding member is configured to provide a surface for manual control of said electrode assembly.

10. The electrode assembly of claim 2, further comprising:
an elongate lead connected to said proximal end of said collar member, said lead physically and electrically connecting said electrode assembly an implanted stimulator unit.

11. The electrode assembly of claim 10, wherein said lead is continuous with no intermediate electrical connectors between said electrode assembly and the stimulator unit.

12. The electrode assembly of claim 1, wherein said plurality of electrodes are positioned along said carrier member so as to align with predetermined regions of a tonotopically mapped cochlea.

13. The electrode assembly of claim 1, wherein said carrier member has opposing medial and lateral longitudinal surfaces, wherein when implanted, said medial surface faces toward the center of the cochlea and said lateral surface that faces toward the exterior of the cochlea,
wherein at least one of said plurality of electrodes has a surface that is collocated with said medial surface of said carrier member.

14. The electrode assembly of claim 1, further comprising:
transmission means for communicably coupling said plurality of electrodes with a stimulator unit.

15. The electrode assembly of claim 1, wherein each of said plurality of electrodes is electrically connected to at least one multi- or single-filament wire embedded within said carrier member.

16. The electrode assembly of claim 15, wherein said wires are embedded in a volumetric core of said carrier member.

17. The electrode assembly of claim 15, wherein said wires are located at or near surfaces of said carrier member.

18. The electrode assembly of claim 15, wherein said wires are located in different regions of said carrier member to facilitate curvature or to maintain orientation of said carrier member once said carrier member is implanted.

19. An electrode assembly for use in a prosthetic hearing implant, comprising:
an elongate carrier member configured for implantation into a cochlea, the carrier member having a proximal end adapted to be positioned in a basal region of the cochlea, and a distal end adapted to be positioned in an apical region of the cochlea;
a plurality of electrodes disposed on said carrier member;
wherein said carrier member has a length such that said carrier member cannot be implanted in the cochlea beyond a maximum insertion depth at which residual hearing of the apical region of the cochlea is substantially preserved and at which said carrier member provides electrical-only stimulation to a depth in the cochlea of approximately 270° to 320°.

20. The electrode assembly of claim 19, further comprising:
a laterally-extending collar member connected to said proximal end of said carrier member and configured to abut the cochlea when said carrier member is implanted at said maximum insertion depth, and to prevent further insertion of said carrier member into the cochlea.

21. The electrode assembly of claim 19, wherein said carrier member is longitudinally-tapered, wherein said proximal end of said carrier member has greater dimensions than said distal end of said carrier member.

22. The electrode assembly of claim 20, wherein in one or more radial directions said collar member has a diameter greater than a diameter of said carrier member.

23. The electrode assembly of claim 20, wherein said collar member has a length of between about 1.5 mm and about 2.5 mm.

24. The electrode assembly of claim 20, further comprising:
a band circumferentially surrounding said proximal end of said carrier member, said band formed of a material that produces a tissue reaction that facilitates tissue attachment to the band.

25. The electrode assembly of claim 19, wherein said carrier member has opposing medial and lateral longitudinal surfaces, wherein when implanted, said medial surface faces toward the center of the cochlea and said lateral surface that faces toward the exterior of the cochlea, wherein said electrode assembly further comprises:
a tapered tip region that slopes from said distal end toward said proximal end, and from said lateral surface inward toward said medial surface.

26. The electrode assembly of claim 20, further comprising:
a holding member having a distal end connected to said proximal end of said collar member, wherein said holding member is configured to provide a surface for manual control of said electrode assembly.

27. The electrode assembly of claim 20, further comprising:
an elongate lead connected to said proximal end of said collar member, said lead physically and electrically connecting said electrode assembly an implanted stimulator unit.

28. The electrode assembly of claim 27, wherein said lead is continuous with no intermediate electrical connectors between said electrode assembly and the stimulator unit.

29. The electrode assembly of claim 19, wherein said carrier member has opposing medial and lateral longitudinal surfaces, wherein when implanted, said medial surface faces toward the center of the cochlea and said lateral surface that faces toward the exterior of the cochlea,
wherein at least one of said plurality of electrodes has a surface that is collocated with said medial surface of said carrier member.

30. The electrode assembly of claim 19, further comprising:
transmission means for communicably coupling said plurality of electrodes with a stimulator unit.

31. The electrode assembly of claim 19, wherein each of said plurality of electrodes is electrically connected to at least one multi- or single-filament wire embedded within said carrier member.

32. The electrode assembly of claim 31, wherein said wires are embedded in a volumetric core of said carrier member.

33. The electrode assembly of claim 31, wherein said wires are located at or near surfaces of said carrier member.

34. The electrode assembly of claim 31, wherein said wires are located in different regions of said carrier member to facilitate curvature or to maintain orientation of said carrier member once said carrier member is implanted.
